(19) United States
(12) Patent Application Publication
(10) Pub. No.: US 2013/0039950 A1
(43) Pub. Date: Feb. 14, 2013

(54) NETI-POT PACKET MIXTURE

(71) Applicants: Yun XU, Langhorne, PA (US); Jennifer Benson, Newtown, PA (US)

(72) Inventors: Yun XU, Langhorne, PA (US); Jennifer Benson, Newtown, PA (US)

(73) Assignee: CHURCH & DWIGHT CO., INC., Princeton, NJ (US)

(21) Appl. No.: 13/205,997

(22) Filed: Aug. 9, 2011

(51) Int. Cl. A61K 9/14; A61P 11/02; B65D 69/00; A61K 33/14

(52) U.S. Cl. 424/400; 424/680; 206/572

(57) ABSTRACT

A nasal rinse composition comprising about 54 to 90 wt. % of sodium chloride and about 10 to 46 wt. % of sodium bicarbonate, and a kit comprising a packet that contains the rinse composition and a nasal rinse dispenser.

### TECHNICAL FIELD

This invention relates to a composition and kit for rinsing nasal passages.

### BACKGROUND

The nose is a very specialized and complicated structure that serves dual functions; it is the organ for the sense of smell, and it is also an entrance to the respiratory tract. As a part of the respiratory tract, a healthy nose moisturizes and warms incoming air and filters out foreign materials.

Nasal passages and other portions of the respiratory tract are lined with specialized tissue layers. In the nose and sinus areas, this tissue is often called the nasal mucosa. Like many tissues, the nasal mucosa is composed of several cell layers and cell types. Mucous cells are located throughout the nasal mucosa and are generally clustered into small glands. These glands secrete a sticky substance called mucus. Mucus is composed of water, shed epithelial (surface) cells, dead leukocytes, mucin, and inorganic salts, among other things, that are all held in suspension. Mucus functions as a trap for airborne particles (e.g., dust, bacteria, and viruses) that enter the nasal passages. Mucus also lubricates the walls of the nose, sinuses, and throat.

In a healthy nose, the mucus is cleared from the nasal passages on a regular basis by a layer of cells in the nasal mucosa called the ciliated columnar epithelium. These cells possess small hair-like projections called cilia that undulate and “sweep” mucus through the nasal passages allowing it to drain to the back of the throat where it can be swallowed or expelled. This line of defense protects the body against the bacteria and viruses that continually enter the nose and mouth.

Connected to the nose are sinuses or air-filled cavities located behind certain facial bones. There are four groups of sinuses, namely: frontal, sphenoidal, ethmoidal, and maxillary. The sinuses are also lined with a mucus-secreting tissue. The sinuses are normally kept clear when mucus drains through them into the nasal passages. If they are obstructed for any reason, such as from the congestion present during a cold, normal drainage may not occur and the sinuses may be subsequently infected.

Virtually all persons are occasionally stricken with acute upper respiratory infections (common colds), acute or chronic allergy flare-ups of the nose, and/or acute or chronic non-allergic rhinosinusitis. Persons afflicted by such conditions usually incur significant discomfort and inconvenience.

All of these disorders are characterized by intense inflammation of the nasal mucosa. A number of symptoms contribute to the discomfort and inconvenience associated with the common cold or sinusitis. Symptoms often include one or more of the following: nasal congestion, post-nasal drip, decreased sense of smell, ear fullness, headache, sore throat, malaise, muscle and joint aches, fatigue, cough, chest congestion, fever, chills and gastrointestinal maladies. Considerable research has been conducted over the years aimed at reducing the incidence and duration of symptoms associated with allergies and common colds.

Perhaps the most common symptom experienced by cold and allergy sufferers is “congestion.” Congestion is commonly used by the layperson and in the patent literature as catch-all term to describe a complex series of events that hinder a person's ability to breathe through the nose when they have a cold, sinusitis or an allergic reaction.

For example, congestion can include a physiological response to an irritant that involves increased blood flow to tissues lining the nose. This increased blood flow causes these tissues to swell and physically block the nasal passages. Alternatively, when a nose is irritated or injured, fluid can seep into the free spaces in the tissues of the nose or build up in individual cells thereby causing additional congestion.

Congestion can also include the overproduction of mucus or the buildup of mucus due to increased mucus viscosity or depressed cilia activity or both. Congestion may also occur when a person lies on his or her side causing receptor cells in the nose to close off one nasal airway. Changes in temperature and/or humidity can alter the tissue of the nose and cause a congested feeling.

There are various methods to treat the symptoms of or to cure sinus disease, including surgery. Saline nasal irrigations have been used for many years and have been mentioned in medical textbooks going back hundreds of years. A wide variety of techniques have been described, including swimming in salt water, which often results in some degree of inadvertent nasal salt water irrigation. Alternatively, an effective nasal rinse can significantly reduce or permanently cure the symptoms of nasal allergies and sinus disease.

Nasal rinsing or lavaging is a treatment for rhinitis and sinusitis. Nasal rinsing is comprised of the dispensing of a saline solution into nasal passages, in order to cleanse and wash away mucus and allergens that create particles and irritants. Nasal rinsing allows the sinuses to drain normally and reduces the inflammation of the mucus membrane.

Saline solutions can be purchased, or prepared at home using household ingredients. However, there is a concern that the ready-to-use saline solutions may be contaminated, and that those solutions may not have a proper concentration of salt and acidity level. In particular, a saline solution made from only table salt is acidic in nature, and this acidity can cause burning when used to rinse nasal passages. Furthermore, there is a need to maintain the saline solution at a pH level consistent with the pH of mucous membranes of tissues, so that the saline solution will not irritate the nasal membrane and cause a stinging and burning sensation.

Numerous examples of nasal solutions, methods of use, and variations thereof have been reported. For example, topical nasal moisturizers and washes may be formulated so as to be isotonic, hypotonic, or hypertonic with respect to saline. Examples of marketed topical nasal moisturizers include NaSal™ Moisturizer, which contains sodium chloride 0.65%, benzalkonium chloride, thimerosal 0.001% as preservatives, mono- and dibasic sodium phosphates as buffers, and purified water.

Another example is Ayr® Saline Nasal Mist and Drops, which is similarly preserved and contains a buffered isotonic saline. Ayr® Saline Nasal Mist and Drops uses monobasic potassium phosphate/sodium hydroxide buffer to adjust the pH and tonicity of the saline. It is desirable to instead provide a product in form of a dry and blended powder to extend its storage time. It is also advantageous to let the consumer to prepare the saline solution with water of a specific source and temperature at his or her choosing.

In another example, U.S. Pat. No. 6,520,384 (Mehta) discloses the use of an apparatus and a saline solution contained therein for rinsing nasal passages. Mixing water with a blend of 39 parts of sodium chloride and 1 to 2 parts of sodium bicarbonate produces the saline solution. However, the pH-consistency issue still persists in this formulation, as the pH can wildly fluctuate based upon the type of water that was used.

Nasal rinsing equipment currently available includes various types of dispensers that can be filled with a saline solution, which are then injected into the user's nasal passages. Conventional nasal rinsing dispensers are often crude, unable to hold a sufficient amount of liquid, and/or lack a proper opening that fits into the user's nostril to dispense the liquid.

For the foregoing reasons, there is a need for a solid blend of salt having a prolong storage time without preservatives. The solid blend can be mixed with any potable water to form a pH-balanced saline solution, such that the solution does not cause a stinging or burning sensation upon its administration into nasal passages. There is also a need for an inexpensive and sturdy dispensing system to prepare and dispense the pH stabled saline solution. The dispensing system may contain a spout for delivering the solution into nostril of various sizes.

### SUMMARY OF THE INVENTION

The present invention is directed toward a composition and a kit that prepares and dispenses a saline solution into nasal passages. The composition is comprised of a solid blend of sodium chloride and sodium bicarbonate, and the kit is comprised of packages of the saline blend and a dispensing assembly to dispense a liquid into nasal passages. The dispensing assembly is comprised of a container known as a neti pot.

### BRIEF DESCRIPTION OF THE DRAWINGS

FIG. 1 is a perspective view of a dispensing assembly and a carton containing multiple packets, each packet comprises a solid blend of sodium chloride and sodium bicarbonate.

### DETAILED DESCRIPTION OF THE INVENTION

The present invention is directed toward a composition and a nasal kit that prepares and dispenses a saline solution into nasal passages. The composition is comprised of a solid blend of sodium chloride and sodium bicarbonate. The nasal kit is comprised of packets of the solid blend, and a dispensing assembly is comprised of a neti pot that has a spout for directing a liquid into nasal passages. The composition is mixed with water to form a saline solution. The composition is comprised of a sufficiently high concentration of sodium bicarbonate, such that it surprisingly provides a consistent pH for the saline solution, regardless the type of water that was used to dissolve and mix the solid blend.

### The Composition

The composition is provided as a solid blend of sodium chloride and sodium bicarbonate, such that the blend can be dissolved and mixed with water to form a saline solution. The solution is subsequently administered to nasal passages.

Sodium chloride (NaCl) is used in the granule form, and is presented in an amount of about 54 to 90 wt. % of the total composition, preferably about 58 to 87 wt. % of the total composition, and more preferably about 61 to 83 wt. % of the total composition.

Sodium bicarbonate (NaCOH₃) is used in either a granule or powder form, but preferably in the same form as sodium chloride. By having the same physical form during the manufacturing process, the separation of the two components can be minimized, and the uniformity of the blend can be maximized. The sodium bicarbonate can be one of the various USP grades selected from 1, 2, 3, 4, 5, MP, or micronized. Grade MP is preferred. The particle size of the sodium bicarbonate powder is between 149 and 250 microns. A coarser particle size is possible. The amount of sodium bicarbonate used is about 10 to 46 wt. % of the total composition, preferably about 13 to 42 wt. % of the total composition, and more preferably about 17 to 40 wt. % of the total composition. The particle size of the granulated sodium chloride is substantially different from the particle size of the sodium bicarbonate.

The mixture of sodium chloride and sodium bicarbonate is uniformly blended prior to its incorporation into a packet or pouch. The mixture has a density and a TAP density of about 1.0 to 1.5 g/cm³.

The packet or pouch can be of any conventional size, for example, 1.5×2.5 in. Typically, the packet has an appropriate size to accommodate about 1.5 to 4.0 g of the mixture, preferably about 2.0 to 3.0 g, and more preferably 2.0 to 2.5 g of the mixture. The packet must have a low moisture permeability and be sealed completely upon filling with the mixture. An aluminum lining may be used inside the packets to protect the contents from moisture, which can adversely affect the ease with which the NaCl/NaHCO₃ dissolves in the water. A dotted line can be marked on the exterior of the packet to provide a guide for cutting open the packet.

### The Saline Solution

Each packet contains a solid blend of approximately 54 to 90 wt. % of NaCl and 10 to 46 wt. % of NaHCO₃, preferably 58 to 87 wt. % of NaCl and 13 to 42 wt. % of NaHCO₃, and more preferably 61 to 83 wt. % of NaCl and 17 to 40 wt. % of NaHCO₃. The blend is dissolved with a sufficient amount of water to produce an isotonic saline solution, which has a concentration of approximately 0.5 to 2% of sodium, preferably 0.7 to 1.5% of sodium, and more preferably 0.9% to 1% of sodium. The water is selected from distilled water, tap water, boiled water and filtered water.

The isotonic saline solution is pH-balanced and compatible with the human nasal and sinus mucosa to prevent burning or stinging during nasal lavaging. Burning or stinging are negative sensations that are often caused by plain water, or by saline solution prepared from either home ingredients, (i.e. table salt) or an ill-proportioned blend of sodium chloride and sodium bicarbonate.

### Dispensing Assembly

The dispensing assembly is comprised of an apparatus that dispenses the saline solution to rinse nasal passages. The apparatus is comprised of a container (neti pot), which has an opening, a spout and a cap. The cap is removable, so that it exposes the opening to allow the neti pot to be filled with the saline solution, or with water and the solid blend to prepare the saline solution. The spout dispenses the saline solution into nasal passages.

The attachment of the cap to the neti pot can be accomplished in any convenient fashion, so that it allows for removability and maintains an adequate liquid seal to prevent leakage during nasal rinse. Methods of attachment range from a ring and groove assembly, a compression-fitted cap, to exterior clamps or the like. The exterior surface of the cap can include rounded, vertical ridges to allow a user to grip the cap when he or she secures the cap onto, or removes the cap from the neti pot.

While the cap is secured to the container, the neti pot can have flexible sidewalls that can be easily compressed by hand to force the saline solution through the spout. Alternatively, the neti pot is inflexible, and the saline solution is dispensed through the spout by gravity. The sidewalls optionally contain a grip portion for the user to grip the container by hand without a handle. The sidewalls alternatively have a handle attachment for the user to grab onto while dispensing the saline solution through the spout. The neti pot can be made of various materials, such as low-density polypropylene, high-density polypropylene, wood, metal, ceramic, or any materials that are sturdy enough to enable the neti pot to be held and shaken by hand, and to retain the saline solution with minimal or no leakage. The neti pot can be transparent, opaque, or solid in appearance. The neti pot should be microwave safe to allow convenient heating of the contents within the neti pot.

The neti pot optionally includes a marking to indicate a liquid level. The marking can be in any convenient form such as a printed line, a groove, a ring or the like. The neti pot holds a minimum of 10 oz. of water, preferably 9 oz. of water, and more preferably 8 oz. of water.

The spout is shaped to fit comfortably into nostril of various sizes. The spout can be formed from the same material as the neti pot, or of a different material.

The assembly optionally includes a plug or stopper (not shown) that fits onto the spout, so that it retains the saline solution in the neti pot during its transportation. The connection of the plug to the spout can be of any convenient means, including a compression-fitted or threaded connection.

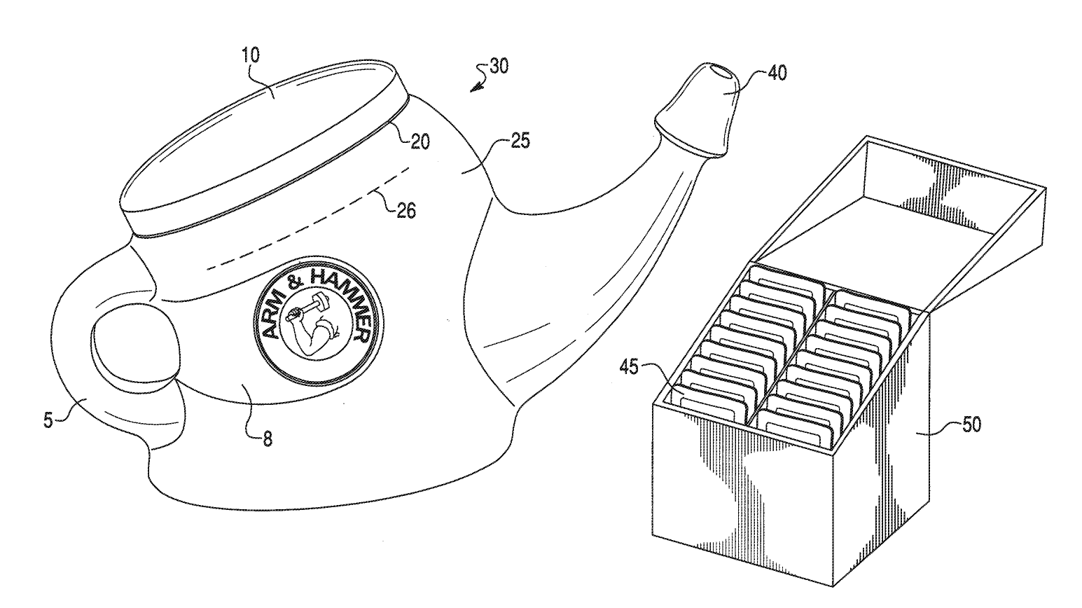

FIG. 1 depicts a nasal kit that is comprised of a dispenser assembly 30 and a carton 50 that includes at least one saline mixture packet 45. The dispenser assembly 30 includes a neti pot 25, a handle 5, a grip area 8, an opening 20, a cap 10, and a spout 40.

### Method of Use

First remove cap 10 removed from neti pot 25, and fill neti pot 25 with 8 oz. of water. The water used is selected from distilled water, tap water, boiled water and filtered water. A dashed line 26 is marked on the exterior of neti pot 25, to indicate to a user when 8 oz. of water has been poured into neti pot 25. The water can then be warmed in a microwave oven. To avoid excessive heating, it is recommended to warm the water using five seconds increments. If the water is heated to hotter than lukewarm, it is recommended that the water to be allowed to cool before proceeding. Alternatively, the water can be warmed before it is poured into neti pot 25, or the water does not have to be warmed at all.

Packet 45 contains a solid blend of NaCl/NaHCO₃. The packet is cut open and the content is emptied into neti pot 25. Cap 10 is secured onto opening 20 and spout 40 is plugged by hand, a plug or a stopper (not shown). Dispenser assembly 30 is then shaken by hand until the NaCl/NaHCO₃ mixture is fully dissolved in the water. The mixture of sodium chloride (NaCl) and sodium bicarbonate (NaHCO₃) in water constitutes a ready-to-use saline solution. The solution has a concentration of approximately 0.9% to 1% of sodium. Another packet 45 can also be added to the saline solution.

The user bends forward to a comfortable position, tilts his or her head slightly up, holds neti pot 25 pointing downward (with opening 20 closed by cap 10 and spout 40 unobstructed) by handle 5 or grip portion 8, and applies spout 40 directly into his or her nostril. With the user's mouth open, neti pot 25 is then moved to force the saline solution to enter the user's nostril and nasal passage. The process is repeated by applying spout 40 snugly against the other nostril. The saline solution that was dispensed into the nasal passages will drain from the nasal passages or the mouth and should not be swallowed by the user. The user then gently blows the nose. Any unused portion of the saline solution is discarded and dispenser assembly 30 is then cleaned. A nasal rinse can be performed as often as needed, or as recommended by a qualified physician.

Dispenser assembly 30 should be thoroughly cleaned after each nasal rinse usage. Dispenser assembly 30 can be placed in a dishwasher to be cleaned, or the assembly can be lathered with light soap then thoroughly rinsed under running water, then left to air-dry in an adequately ventilated area. Additionally, a vinegar and water solution can also be used to clean dispenser assembly 30.

### Experimental Data

Four packets of equally proportioned, solid blend of sodium chloride and sodium bicarbonate (labeled “Testing Mix”) were prepared, with an identical amount of 27.8 wt. % of sodium bicarbonate (NaHCO₃). Each packet was dissolved and mixed separately in 8 oz. of lukewarm distilled water, tap water, boiled water, or filtered water. Similarly, four packets of equally proportioned NeilMed®, each contained 3.4% sodium bicarbonate were separately dissolved and mixed in 8 oz. of distilled water, tap water, boiled water, or filtered water. Four equally portioned Ayr® Nasal Rinse were also dissolved in the same fashion.

The pH of each of the dissolved sample was measured and the result is shown below:

| | | pH | | | |
|---|---|---|---|---|---|
| Water Temp | Type of | Distilled | Tap | Boiled | Filtered |
| (32.5 C.) | Liquid | Water | Water | Water | Water |
| Testing Mix | Pure Water | 5.87 | 7.44 | 8.71 | 4.23 |
| (Contains 27.8% | Water + | 8.08 | 8.03 | 8.12 | 7.72 |
| NaHCO₃) | Testing Mix | | | | |
| NeilMed ® | Pure Water | 5.87 | 7.44 | 8.71 | 4.23 |
| (Contains 3.4% | Water + | 8.08 | 7.79 | 8.31 | 6.71 |
| NaHCO₃) | NeilMed | | | | |
| Ayr ® (Contains | Pure Water | 5.87 | 7.44 | 8.71 | 4.23 |
| 0.5% NaHCO₃) | Water + Ayr | 7.61 | 7.45 | 8.32 | 5.77 |

As shown, the pH for distilled water, tap water, boiled water and filtered water ranged from 8.71 to 4.23. When one packet of the Testing Mix was dissolved in 8.0 oz. of each of the above four types of water, it brought the pH range of the solutions to between 8.03 and 7.72, which is narrower than the ranges of the pure water and the competitors' products that contained less sodium bicarbonate.

1. A nasal rinse composition, comprising about 54 to 90 wt. % of sodium chloride and about 10 to 46 wt. % of sodium bicarbonate.

2. The nasal rinse composition of claim 1, wherein said sodium chloride comprises about 58 to 87 wt. % and said sodium bicarbonate comprises about 13 to 42 wt. % of said nasal rinse composition.

3. The nasal rinse composition of claim 2, wherein said sodium chloride comprises about 61 to 83 wt. % and said sodium bicarbonate comprises about 17 to 40 wt. % of said nasal rinse composition.

4. The nasal rinse composition of claim 1, comprising a solid blend of said sodium chloride and sodium bicarbonate.

5. A nasal rinse composition of claim 4, wherein said sodium bicarbonate has a particle size of between 149 and 250 microns.

6. A nasal rinse kit comprising 1) at least one package comprising a nasal rinse composition comprising about 54 to 90 wt. % of sodium chloride and about 10 to 46 wt. % of sodium bicarbonate, and 2) a dispensing assembly for mixing said nasal rinse composition with water to form a saline solution, and delivering said saline solution to nasal passages.

7. A nasal rinse kit of claim 6, wherein said saline mixture comprises sodium chloride at an amount of about 58 to 87 wt. % and sodium bicarbonate in an amount of about 13 to 42 wt. % of the total composition.

8. A nasal rinse kit of claim 7, wherein said saline mixture comprising sodium chloride at an amount of about 61 to 83 wt. % and sodium bicarbonate in an amount of about 17 to 40 wt. % of the total composition.

9. The nasal rinse kit of claim 6, wherein said nasal rinse composition comprises a solid blend of said sodium chloride and sodium bicarbonate.

10. A nasal rinse kit of claim 9, wherein the particle size of said sodium bicarbonate is of between 149 and 250 microns.

11. A nasal rinse kit of claim 6, wherein said dispensing assembly comprises a neti pot.

12. A nasal rinse kit of claim 11, wherein said neti pot comprises a cap, an opening, a spout and optionally a stopper for said spout.

13. A nasal rinse kit of claim 11, wherein said neti pot further includes a marking to indicate a liquid level.

14. A method for rinsing nasal passages, comprising 1) pouring a solid saline blend from a package comprising about 54 to 90 wt. % sodium chloride and about 10 to 46 wt. % sodium bicarbonate into a dispensing assembly; 2) mixing said blend with water in said dispensing assembly to form a saline solution; and 3) delivering said saline solution to nasal passages from a spout of said dispensing assembly.
